(19) United States
(12) Patent Application Publication
(10) Pub. No.: US 2014/0172028 A1
(43) Pub. Date: Jun. 19, 2014

(54) SURFACE TREATMENT PROCESS FOR IMPLANTABLE MEDICAL DEVICE

(71) Applicant: Neil Meredith, Harrogate (GB)

(72) Inventor: Neil Meredith, Harrogate (GB)

(73) Assignee: Neoss Limited, Harrogate, Yorkshire (GB)

(21) Appl. No.: 14/233,485

(22) PCT Filed: Jul. 18, 2012
(86) PCT No.: PCT/GB2012/051710

§ 371 Date: Jan. 17, 2014

(30) Foreign Application Priority Data
Jul. 19, 2011 (GB) 1112407.0

(51) Int. Cl. A61C 8/00; A61B 17/86

(52) U.S. Cl. CPC A61C 8/0015; A61B 17/866
USPC 606/331; 427/2.26

(57) ABSTRACT

A surface treatment process for improving the hydrophilicity of at least part of an implantable medical device. The process comprises applying to the surface of the implantable medical device a solution of a non-ionic substance having at least one polar covalently-bonded group and drying the implantable medical device to form a hydrophilic deposition of the substance on the surface of the implantable medical device.

### FIELD OF THE INVENTION

This invention relates to the field of surface treatment processes for implantable medical devices and medical devices treated with such processes.

### BACKGROUND

In many types of implanted medical device (for example, orthopedic implants, pedicle screws, dental implants, spinal implants and sensors) it is desirable to have a strong interaction between the surface of the device and the surrounding tissues (i.e. bone for an endosseous or bone-anchored implant) for the purpose of load and stress transmission, as well as to prevent bacterial ingress, infection and inflammation. Such devices are used to stabilize fractures, strengthen weak bones and anchor prostheses.

The surfaces of such devices (hereafter referred to generally as “implants”) have been shown to osseointegrate when surrounded by bone. Osseointegration (the formation of a structural connection between the implant and the surrounding living bone) occurs following implant placement as a result of new bone formation or remodelling of the existing bone which is in direct contact with the implant's surface. Such osseointegration has been demonstrated in many studies histologically, radiographically and with pull out, removal torque, resonance frequency analysis and other mechanical tests.

Implants are typically pure metals, alloys or ceramic devices. Titanium and its alloys, zirconia, hafnium, tantalum, stainless steel, hydroxyapatite and cobalt chromium are commonly-used materials. It is well understood that the surface topography (roughness, surface characterization whether random or repeated) of the implant may influence the rate and quality of bone formation at the implant-tissue interface. In general, it is considered that implants which have been roughened on the nanometer and micrometer scale can achieve the maximal bone to implant contact and increase the rate and quality of bone formation. The consequent reduction in the time taken for healing and osseointegration is highly desirable, enabling early loading and reduced treatment times. In addition the strength and stiffness of the implant-bone interface can be greater with surfaces having certain topographies.

There are a number of well documented methods for the alteration of the surface topography or roughness of implants. These may include particle blasting (grit, sand and other abrasive particles), acid etching, plasma spraying, anodizing, micro-arc oxidation or a combination of these. This may result in a single level of roughness or multiple modulated levels of roughness ranging from a scale of 1 nm to 100 μm. Topography and textures of these types are well known from commercial products and for example from EP 0 388 576.

The surface modification processes described above can also alter the chemistry of the surface. Typically metals form surface oxides on exposure to air and water. Such exposure may occur during production or surgical placement or handling. Chemically, the surface of the implant may be the metal itself, an oxide of the metal, for example titanium or titanium oxide. Carbon and other impurities may be present on the implant surface as a result of the production, storage or handling procedures.

It is highly desirable that, when an implant is placed into the tissues or bone, it is thoroughly wetted with the body's natural tissue fluids. Tissue fluids contain nutrients, electrolytes, proteins, growth factors and other substances essential in the healing and bone formation process. Implants may also be pre-treated with liquids or gels, growth factors for example during production or prior to treatment. Any liquid, gel or solution contacting an implant should thoroughly wet the surface and penetrate any surface roughness or topographical features.

It has been shown that there is a correlation between biocompatibility, bioadhesion and surface tension or contact angle on a substrate or implant surface (Baier, 1972, The role of surface energy in thrombogenesis, Bull. N. Y. Acad. Med. 48, 257-272). One of the major problems with implants having roughened surfaces is the hydrophobicity or inability of the surface to wet adequately when liquids are applied to it. This may be due to contamination of the surface with organic or hydrophobic material or to the geometry of the surface preventing penetration of fluid due to bridging or surface tension. Bridging is a feature of the aspect ratio of the width to the depth of an area of roughness on a surface. Wetting, hydrophilicity and hydrophobicity of surfaces measured as the contact angle can readily be deduced using a goniometer or Wihelmy plate. A low contact angle of 10° or less occurs on hydrophilic surfaces; a higher contact angle indicates a hydrophobic surface. Dental implants generally have hydrophobic surfaces.

It is essential that tissue fluids or applied liquids penetrate the topography of a surface completely to ensure that nutrients, proteins and growth factors can maintain cell metabolism, healing and bone formation. However the nature of the topography or texture of the surface is important. Increasing the roughness of a surface may cause bridging or air to be trapped under a liquid layer preventing wetting. In addition, the aspect ratio (height or depth of troughs or porosities in relation to their width or circumference) of the topography is critical as this may cause bridging and bridge formation with a failure of a fluid to penetrate such features.

The document WO-A-2010/094968 describes a surface treatment process for an implantable medical device including a surface dielectric insulating layer, where the process involves applying ions onto the dielectric insulating layer. The electrical charge created when the ions in combination with dielectric material are exposed to a liquid eliminates bridging effects and hence reduces surface tension. This electrowetting process increases the hydrophilicity or wettability of the implant surface. In one described example, the electric field may be used to attract specific biomolecules to the dielectric insulating layer.

The present invention, at least in the presently preferred embodiments, seeks to provide an alternative surface treatment process for improving the hydrophilicity or wettability of an implantable medical device.

### BRIEF SUMMARY OF THE DISCLOSURE

In accordance with the present invention there is provided a surface treatment process for improving the hydrophilicity of at least part of an endosseous implantable medical device. The process comprises applying to a surface of the implantable medical device a non-ionic substance having at least one polar covalently-bonded group, for example a sugar with a hydroxyl group, whereby to form a stable deposition of the substance on the surface of the implantable medical device, the deposition being capable of increasing the hydrophilicity of the surface upon application of a liquid thereto by attracting polar water molecules contained in the liquid. The hydroxyl group attracts water because of the electronegativity of the oxygen atom which makes the functional group polar. This polarity attracts the also polar H₂O water molecule creating a hydrophilic effect.

Thus, in accordance with the present invention, the surface of the implantable medical device is modified by application of a non-ionic substance having at least one polar covalently-bonded group. It has been found that the use of a non-ionic substance having at least one polar covalently-bonded group can improve the hydrophilicity or wettability of the surface of the device. The non-ionic substance can also improve the surface appearance of the implantable device and may ensure that the appearance of the surface is maintained during storage of the treated device.

The substance is “non-ionic” in the sense that the atoms forming the molecules of the substance are covalently bonded, rather than ionically bonded. As used herein, “polar covalently-bonded” refers to a separation of electric charge across the bond so that the molecule of the non-ionic substance has an electric dipole moment or multipole moment. For example, the elements forming the polar-covalent bond may have a difference in electronegativity of between 0.4 and 1.7 with respect to one another. One of the elements forming the polar-covalent bond may be particularly electronegative, and may, for example, be one of nitrogen, oxygen or fluorine.

In one embodiment of the invention, the at least one polar covalently-bonded group comprises a polar hydroxyl (—OH) group. The hydroxyl group(s) of the substance exhibit excellent hydrophilicity.

In embodiments of the invention, the substance used in the process of the invention is sufficiently soluble to form a solution for application to the surface of the implantable medical device. In addition, the substance may form a solid deposition or coating on the surface of the device. In particular the substance may be crystalline in its solid form. The deposition of the substance may form a coating on the surface of the device. The coating may cover the entire surface or a portion or portions thereof. The non-ionic substance need not cover the entire surface of the implantable medical device and does not form any protective coating function in the conventional sense.

In a presently preferred embodiment, the substance is a carbohydrate. The carbohydrate may be a sugar or a sugar alcohol. Sugars and sugar alcohols are generally biologically acceptable and readily available. The sugar may be a monosaccharide or a disaccharide. Sugars of higher molecular weight will generally not exhibit desirable solubility for use in the process of the present invention. Examples of suitable sugars include fructose, maltose, dextrose, glucose or sucrose, or combinations thereof. A sugar alcohol such as xylitol may also be used as the substance of the present invention.

The non-ionic substance may be applied to a surface of the implantable medical device in solution. Typically, the solution is an aqueous solution. However, alcohol or other solvents may be used. The solution may be applied to the implantable medical device by any suitable method, for example by spraying, or by immersing or dipping the device into the solution. The solution may be applied to the device by immersing the device in an ultrasonic bath filled with the solution. The device may be immersed in the bath for between 0.1 and 5 minutes. In the presently preferred embodiment, the device is immersed in the bath for 3 minutes. Alternative methods of applying the substance to the surface of the implantable medical device may be used, for example sputtering and plasma vapour deposition.

The device may be mounted on a holder for application of the solution. The holder may be a PTFE holder.

In embodiments of the invention, the solution contains between 1% and 20% by weight of the substance. In particular the solution may contain at least 5% by weight of the substance. The solution may contain less than 15% by weight of the substance. One example solution contains 10% by weight of fructose in water.

The implantable medical device may be dried after application of the non-ionic substance to form the stable deposition of the substance on the surface of the implantable medical device. In embodiments of the invention, the device may be dried at a temperature between 30 and 80 degrees centigrade. The drying temperature may be at least 40 degrees centigrade. The drying temperature may be less than 70 degrees centigrade. A presently preferred drying temperature is 50 degrees centigrade.

The device may be dried for between 5 and 20 minutes. The drying time may be less than 15 minutes. A presently preferred drying time is 10 minutes. Any suitable drying method may be used. In the presently preferred embodiment, a forced convection oven is used.

The process of the invention may include additional steps for preparing the surface of the implantable medical device before the non-ionic substance is applied. For example, the device may be cleaned in an ultrasonic bath of distilled water and air dried prior to application of the substance.

Prior to application of the substance to the implantable medical device, the surface of the device may be chemically etched and/or roughened, for example by particle blasting, such as grit blasting.

The implantable medical device may be composed of metal. The device may be composed in its entirety of metal. Alternatively, at least the surface of the implantable medical device may be metal. A presently preferred metal is titanium.

In the presently preferred embodiment, the implantable medical device is a dental implant.

The present invention extends to an endosseous implantable medical device treated in accordance with the surface treatment process of the invention. In addition, the present invention extends to an implantable medical device having a surface deposition of a non-ionic substance having at least one polar covalently-bonded group.

### BRIEF DESCRIPTION OF THE DRAWINGS

FIG. 1 shows a dental implant for processing in accordance with the invention.

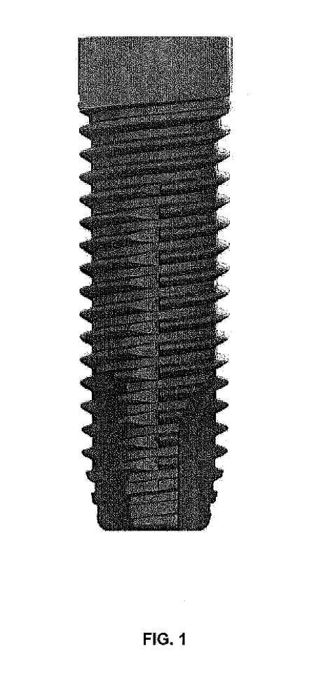

### DETAILED DESCRIPTION

The surface treatment process according to the present invention is for improving the hydrophilicity or wettability that an implantable medical device (“implant” hereinafter), such as a dental implant, exhibits during its insertion or placement into the body. The implant may comprise a metallic or metallic alloy body. The implant body may have a grit blasted and etched, or otherwise textured, surface to improve osseointegration of the implant, and reduce healing time for the patient. The implant may have a metal oxide layer on the surface thereof, for example such as titanium oxide which is commonly present on titanium medical implants. The metal oxide layer may have a thickness in the range 1 nm to 100 μm.

The process of the present invention comprises applying molecules of a substance onto the surface of the implant, where each molecule has at least one polar-covalently bonded group. It is found that the application of such molecules to the implant improves the hydrophilicity of the implant, such that when a liquid is applied to the implant, the measured contact angle is less than 10 degrees.

Liquid in the form of bodily fluid, for example blood or tissue fluid, is applied to the surface of the implant during its placement into the desired implantation site. The hydrophilicity of the implant is improved by the presence of the surface deposition containing molecules having at least one polar covalently-bonded group.

In one example, the molecules are sugar molecules, and may comprise any combination or mixture of fructose, maltose, dextrose, glucose, sucrose or xylitol molecules. Indeed, any monosaccharide or disaccharide (or combination thereof) may be a suitable sugar(s). Of course, the molecules for treating the implant may be exclusively of a single type. Sugars are a particularly preferable choice for the molecules since, on application of a liquid, such as when the implant is implanted into human tissue, the sugar will dissolve completely in the fluid, whether it be saline, blood or other tissue fluid. Sugars, particularly fructose, also provide the treated implants with a desirable surface finish. In addition to their biocompatibility and solubility in water, sugars are only minimally hygroscopic, so the surface finish of the treated implants is stable over time, thereby improving the shelf-life and reliability of the treated implants.

The molecules are applied to the implants in an aqueous or alcohol based solution, where the solution is applied to at least part of the surface layer by spraying, immersing or dipping the device into the aqueous solution. Alternatively, any suitable deposition method may be employed to apply the molecules to the surface layer.

In one embodiment, the molecules are fructose molecules and are further preferably part of an aqueous solution containing between 1% and 10% by weight fructose molecules. In specific examples, the aqueous solution is a 1%, 5% or 10% fructose solution.

In one embodiment, the implant is mounted on a polytetrafluoroethylene (PTFE) holder and cleaned in an ultrasonic bath of distilled water. The cleaned implant is then air dried and the dried implant is placed in an ultrasonic bath filled with the aqueous solution containing the molecules (e.g. 1% aqueous fructose solution) for a predetermined time, such as 3 minutes, for example. The implant is then dried (preferably with its tip pointing upwardly) in a forced convection oven for 10 minutes at 50 degrees centigrade, for example. The treated implant may then be implanted into a patient, or stored for future implantation.

### EXAMPLE

The following is an example of a method for treating dental implants in accordance with one embodiment of the surface treatment process of the present invention.

Method:

1. A batch of dental implants were mounted on polytetrafluoroethylene (PTFE) holders, cleaned in an ultrasonic bath of distilled water and then air dried;

2. The dry implants were then placed in an ultrasonic bath filled with a 1% aqueous solution of fructose for 3 minutes;

3. The implants were then dried (with their tip pointing upwards) in a forced convection oven for 10 minutes at 50 degrees centigrade;

4. Steps 1 to 3 were repeated for separate batches of dental implants using 5% and 10% fructose solutions.

### Results:

All samples exhibited excellent wetting, when tested. Specifically, the measured contact angle on all samples was 0 degrees. Moisture analysis and conductivity analysis of the samples revealed that the implants treated with 1% fructose solution contained 0.247% moisture and 0 ppm of ionic charge carriers. The implants treated with 5% fructose solution contained 0.244% and 0 ppm of ionic charge carriers, and the implants treated with 10% fructose solution contained 0.0% moisture and 0 ppm of ionic charge carriers. Additionally, it was noted that 24 hours after treatment, none of the implant samples exhibited a noticeable change in visual appearance, thereby exhibiting good stability in normal atmospheric conditions.

In summary, there is disclosed herein a surface treatment process for improving the hydrophilicity of at least part of an endosseous implantable medical device. The process comprises applying to the surface of the implantable medical device a solution of a non-ionic substance having at least one polar covalently-bonded group and drying the implantable medical device to form a deposition of the substance on the surface of the implantable medical device. The deposition, upon contact with liquid (e.g. bodily fluid during implantation of the device) increases the hydrophilicity of the surface by attracting thereto polar water molecules contained in the liquid.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A surface treatment process for improving the hydrophilicity of at least part of an endosseous implantable medical device, the process comprising:
applying to a surface of the implantable medical device a non-ionic substance having at least one polar covalently-bonded group, whereby to form a stable deposition of the substance on the surface of the implantable medical device, the deposition being capable of increasing the hydrophilicity of the surface upon application of a liquid thereto by attracting polar water molecules contained in the liquid.

2. The surface treatment process of claim 1, wherein the at least one polar covalently-bonded group comprises a polar hydroxyl (—OH) group.

3. The surface treatment process of claim 1, wherein the substance is a carbohydrate.

4. The surface treatment process of claim 3, wherein the carbohydrate is a sugar or a sugar alcohol.

5. The surface treatment process of claim 4, wherein the sugar is a monosaccharide or a disaccharide.

6. The surface treatment process of claim 5, wherein the sugar is fructose, maltose, dextrose, glucose or sucrose, or a combination thereof.

7. The surface treatment process of claim 4, wherein the sugar alcohol is xylitol.

8. The surface treatment process of claim 1, wherein the non-ionic substance is applied to the surface of the implantable medical device in solution.

9. The surface treatment process of claim 8, wherein the solution is an aqueous solution.

10. The surface treatment process of claim 8, wherein the solution is applied to the implantable medical device by spraying, or by immersing or dipping the device into the solution.

11. The surface treatment process of claim 8, wherein the solution contains between 1% and 20% by weight of the non-ionic substance.

12. The surface treatment process of claim 8, wherein the implantable medical device is dried after application of the non-ionic substance to form the stable deposition of the substance on the surface of the implantable medical device.

13. The surface treatment process of claim 12, wherein the device is dried at a temperature between 30 and 80 degrees centigrade.

14. The surface treatment process of claim 12, wherein the device is dried for between 5 and 20 minutes.

15. The surface treatment process of claim 1, wherein the implantable medical device is composed of metal or metal alloy.

16. The surface treatment process of claim 13, wherein the implantable medical device is composed of titanium.

17. The surface treatment process of claim 13, wherein the implantable medical device is composed of ceramics.

18. The surface treatment process of claim 1, wherein the implantable medical device is a dental implant.

19. (canceled)

20. An endosseous implantable medical device having a surface deposition of a non-ionic substance having at least one polar covalently-bonded group.

21. An endosseous implantable medical device having a surface deposition of a non-ionic substance having at least one polar covalently-bonded group treated in accordance with the surface treatment process of claim 1.

22. (canceled)
